(19) United States
(12) Patent Application Publication
(10) Pub. No.: US 2004/0136961 A1
(43) Pub. Date: Jul. 15, 2004

(54) Nanoparticulate composition for efficient gene transfer

(76) Inventors: Ales Prokop, Nashville, TN (US); Jeffrey M. Davidson, Nashville, TN (US); Gianluca Carlesso, Nashville, TN (US); Derya Unutmaz, Nashville, TN (US)

Correspondence Address: Benjamin Aaron Adler, ADLER & ASSOCIATES, 8011 Candle Lane, Houston, TX, 77071, US

(21) Appl. No.: 10/609,722

(22) Filed: Jun. 30, 2003

Related U.S. Application Data
(63) Continuation-in-part of application No. 10/356,139, filed on Jan. 31, 2003.
Application No. 10/356,139 is a continuation-in-part of application No. 09/169,459, filed on Oct. 9, 1998, now Pat. No. 6,726,934.
(60) Provisional application No. 60/062,943, filed on Oct. 9, 1997.

(51) Int. Cl. A61K 48/00

(52) U.S. Cl. 424/93.2

(57) ABSTRACT

The present invention provides compositions comprising a water-based core solution and a water-based corona solution surrounding the core solution. The compositions comprise polyanionic polymers and salts and polycationic polymers and cations and is useful for adenoviral delivery of a gene or delivery of another drug. The compositions may be nanoparticulate, microcapsular or form a polymeric sheet. Also provided are methods of use for the compositions.

### CROSS-REFERENCE TO RELATED APPLICATION

This application is a continuation-in-part patent application of U.S. Ser. No. 10/356,139, filed Jan. 23, 2003, which is a continuation-in-part patent application of non-provisional application U.S. Ser. No. 09/169,459, filed Oct. 9, 1998, which claims benefit of provisional application U.S. S. No. 60/062,943, filed Oct. 9, 1997, now abandoned.

### BACKGROUND OF THE INVENTION

1. Field of the Invention

The present invention relates generally to the fields of pharmaceutical sciences, protein chemistry, polymer chemistry, colloid chemistry, biomedical engineering and gene therapy. More specifically, the present invention relates to a nanoparticulate composition for gene delivery into resilient cells.

2. Description of the Related Art

Microparticulate systems are particles having a diameter of 1-2,000 μm (2 mm) or, more preferably, 100-500 μm such as the diameter of microcapsules. Nanoparticulates range from 1-1000 nm (0.001-1.0 μm), preferably 10-300 nm. Collectively, these systems are denoted as drug delivery vehicles. All these vehicles can be formed from a variety of materials, including synthetic polymers and biopolymers such as proteins and polysaccharides, and can be used as carriers for drugs and other biotechnology products, such as growth factors and genes.

In the controlled drug and antigen delivery area microparticles and nanoparticles are formed in a mixture with molecules to be encapsulated within the particles for subsequent sustained release. A number of different techniques are routinely used to make these particles from synthetic or natural polymers, including phase separation, precipitation, solvent evaporation, emulsification, and spray drying, or a combination thereof (1-5). Such particulate delivery systems have been widely used, but difficulties with biocompatibility, particle strength and the inability to define and modify parameters critical for such delivery vehicles has prevented this technology from achieving its full potential. A typical problem is a use of organic solvents for manufacturing particles, rather loose association of plasmid DNA within a liposome (6) or a low stability of the DNA-spermine complex at physiological conditions (7). In addition, liposomes exhibit a very low incorporation of highly hydrophilic substances, such as DNA or polynucleotide.

Recent advances in the understanding of gene transfer have attracted tremendous attention during the last two decades. The principal reason for the incredible growth of gene delivery technology is the realization that the best prospect for achieving substantial improvements over current therapies. This prospect is hampered by enormous barriers that a gene construct must overcome before it reaches its target site within the body where it can perform its biological role.

A major subset of existing drug delivery systems, i.e., those based on synthetic polymers, have attracted significant attention as they appear particularly promising (8). This includes polymers, which are inherently biologically active, polymer-drug conjugates, polymeric micelles, nanoparticles and polymer-coated liposomes. A growing number of such formulations are approved by the regulatory authorities in North America, Europe and Asia for clinical use in treatment of cancer, infectious and genetic diseases.

Polymers have several fundamental properties useful in solving gene delivery problems. First, polymers are large molecules that can be designed to be intrinsically multifunctional and thus can be combined either covalently or non-covalently with gene constructs to overcome multiple problems such as, inter alia, solubility, stability and permeabitity. Second, polymers can be combined with various targeting vectors to direct drugs to specific sites in the body. Finally, polymers are ideal for design of controlled and sustained release of the gene at the site of the action.

The success of gene therapy is largely dependent on the development of the gene delivery vector. Recently, gene transfection into target cells using naked DNA, which is a simple and safe approach, has been improved by combining several physical techniques, for example, electroporation, gene gun, ultrasound and hydrodynamic pressure. Chemical approaches have been utilized to improve the efficiency and cell specificity of gene transfer. Several functional polymers that enable controlled release of DNA have also been reported. Construction of a long-lasting gene expression system is also an important theme for nonviral gene therapy. To date, tissue-specific expression, self-replicating and integrating plasmid systems have been reported. Improvement of delivery methods together with intelligent design of the DNA itself has brought about large degrees of enhancement in the efficiency, specificity and temporal control of nonviral vectors.

Development of an efficient method for introducing a therapeutic gene into target cells in vivo is the key issue in treating genetic and acquired diseases by gene therapy. To this end various nonviral vectors have been designed and developed with some of them in clinical trials. The simplest approach is naked DNA injection into local tissues or systemic circulation. Physical, e.g., gene gun or electroporation, and chemical, e.g., cationic lipid or polymer, approaches also have been utilized to improve the efficiency and target cell specificity of gene transfer by plasmid DNA (9).

After administration, however, nonviral vectors encounter many hurdles that result in diminished gene transfer in target cells. Cationic vectors sometimes attract serum proteins and blood cells when entering into blood circulation, which results in dynamic changes in their physicochemical properties. To reach target cells nonviral vectors must pass through the capillaries, avoid recognition by mononuclear phagocytes, emerge from the blood vessels to the interstitium, and bind to the surface of the target cells. They then need to be internalized, escape from endosomes and subsequently find a way to the nucleus while avoiding cytoplasmic degradation. Many barriers in gene transfer and development of vectors exist.

Non-viral gene delivery systems at therapeutic doses require high concentrations of the particles. Positively charged particles readily aggregate as their concentration increases and are quickly precipitated above their critical flocculation concentration. To circumvent this problem hydrophilic polymers like polyethylene glycol (PEG) have been used to create PEGylated particles to provide steric stabilization. The ability to prepare well-defined particles of known and uniform morphology at high concentration is essential to the development of a concentration of Dnase I that results in extensive degradation of free DNA (10).

In addition to formulation stability, storage stability is necessary to provide a practical “bedside” medicine. Lyophilization is a viable method of preparing non-viral gene delivery systems for storage. Lyophilization of nanoparticles has been reported (11). Thus, recent successes in preparing highly concentrated, stable colloidal dispersions that can be lyophilized and re-suspended without loss of gene delivery efficiency suggest that non-viral systems do have a high potential for translation into commercially viable pharmaceutical products.

Until recently, the cells of haematopoietic origin, e.g., dendritic cells and monocytes, were not considered good adenoviral (AdV) targets, primarily because the lack the specific AdV receptors required for productive and efficient AdV infections. In addition, because of limitations inherent in AdV infections, such as short-term expression and a non-integrating nature, their application has been precluded from haematopoietic stem cell (HSC) and bone marrow transduction protocols where long-term expression has been required. With recent insights into the critical interactions between adenovirus (AdV) and cells, new AdV-mediated gene transduction strategies have now been reported that may overcome these limitations.

These new strategies include AdV possessing synthetic polymer coatings, genetically modified capsid proteins or antibody-redirected fibres that can efficiently redirect and retarget AdV to transfer genes in HSC. Furthermore, new hybrid AdV's engineered with both modified capsid proteins also are being developed which can efficiently deliver and integrate AdV delivered genes into HSC. Nevertheless, problems, drawbacks and limitations persist in developing effective gene delivery systems for some medical applications. Literature data indicate that many cells are resistant to gene transfer.

The inventors have recognized an increasing need in the art for effective compositions to stably deliver genes or other drugs in vivo. The prior art is deficient in the lack of nanoparticulate delivery systems for gene or other drug compositions. Specifically, the prior art is deficient in nanoparticulate compositions comprising a drug or an adenoviral vector for gene delivery in vivo. The present invention fulfills this longstanding need and desire in the art.

### SUMMARY OF THE INVENTION

The present invention is directed to a composition or a pharmaceutical composition thereof comprising a water-based core solution and a water-based corona solution surrounding the core. The core solution may comprise polyanionic polymers and an adenoviral polynucleotide construct. Optionally, the core solution may comprise a monovalent or divalent salt, a crosslinking agent or a combination threof. The corona solution may comprise polycationic polymers and cation(s) and, optionally, further may comprise a targeting conjugate.

The present invention also is directed to a similar composition or a pharmaceutical composition thereof where the core solution may comprise sodium alginate, cellulose sulfate, an adenoviral gene construct, sodium chloride or calcium chloride, and optionally, dextran polyaldehyde. The corona solution may comprise spermine hydrochloride, PMCG hydrochloride, pluronic F-68, and, optionally, a dextran-conjugated lectin or a dextran-conjugated glycan.

The present invention is directed further to another composition or a pharmaceutical composition thereof where the core solution may comprise pentasodium tripolyphosphate, kappa (iota)-carrageenan, an adenoviral gene construct and, optionally, sodium chloride or dextran polyaldehyde or a combination thereof. The corona solution may comprise chitosan glutamate, pluronic F-68, and calcium chloride and, optionally, sodium chloride. Furthermore, the corona solution may comprise a dextran-conjugated lectin or a dextran-conjugated glycan.

The present invention is directed further still to methods of delivering a polynucleotide or a drug to a human or a non-human animal to treat a pathophysiological state therein comprising administering the compositions described herein to the human or the non-human animal where the compositions contain a pharmacologically effective amount of the polynucleotide or of the drug.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 demonstrates in vivo gene expression. DNA-ID represents intradermal injection of naked DNA solution (plasmid); Lipofect/DNA is DNA complexed with Lipofectamine reagent (Gibco, Gaithersburg, Md.); and NP/DNA is DNA encapsulated in nanoparticles.

FIGS. 2A-2B demonstrate gene transfer into UMR cells, 1-6 days post-infection (FIG. 2A), normalized per protein (FIG. 2B), as compared to free AdV.

FIGS. 3A-3C show gene transfer of luciferase/green fluorescent protein into CT26 cells with nanoparticular mediated adenoviral-gene transfer or free adenoviral-gene transfer, 1-4 days post-infection, relative units, as compared to free AdV (FIG. 3A). Green fluorescent protein expression at 36 hours post infection by free AdV (FIG. 3B) or NP-mediated AdV (FIG. 3C) is shown.

FIG. 4 demonstrates gene transfer into HT1080 cells, 1-10 days post-infection, normalized per protein, as compared to free AdV.

FIGS. 5A-5B demonstrate gene transfer into BC-1 cells (FIG. 5A) and BC-BL-1 cells (FIG. 5B), at 25 and 37° C., relative units, 1-4 days post-infection.

FIGS. 6A-6B demonstrate gene transfer into mouse islets, 1-2 days post-infection, as compared to free AdV, normalized per protein, as compared to free AdV (FIG. 6A) and as compared to free AdV, normalized per DNA, as compared to free AdV (FIG. 6B).

FIG. 7 demonstrates gene transfer into dendritic cells, 3 days post-infection, relative units, as compare to free AdV. NPs-AV: nanoparticles loaded with adenovirus (AV); AV: free adenovirus; empty NPs: no adenovirus; cond. media: only conditioned medium used.

### DETAILED DESCRIPTION OF THE INVENTION

In one embodiment of the present invention there is provided a composition comprising a water-based core solution which itself comprises polyanionic polymers and an adenoviral polynucleotide construct or a drug and, optionally, a monovalent or divalent salt or a cross-linking agent or a combination thereof; and a water-based corona solution surrounding the core solution with the corona solution comprisomg at least one cation; polycationic polymers; and optionally, a targeting conjugate; or a pharmaceutical composition thereof.

In all aspects of this embodiment, the monovalent or divalent salt is sodium chloride, calcium chloride or sodium sulfate. A representative crosslinking agent is dextran polyaldehyde and a representative targeting conjugate is a dextran-conjugated lectin or a dextran-conjugated glycan. The polynucleotide may be a gene. A representative example of a gene is one expressing an antiangiogenic growth factor. Examples of the drug are antiangiogenic growth factors, such as endostatin or thrombospondin 1 and 2 or a combination thereof.

Further to all aspects of this embodiment the polyanionic polymers may be sodium alginate, pentasodium tripolyphosphate, kappa (iota) carrageenan, low-esterified pectin, polyglutamic acid, cellulose sulfate or chondroitin sulfate. The polycationic polymers may be polyvinylamine, spermine hydrochloride, protamine sulfate, polyethyleneimine, polyethyleneimine-ethoxylated, polyethyleneimine-epichlorhydrin modified, quarternized polyamide, polydiallyldimethyl ammonium chloride-co-acrylamide, chitosan glutamate, or pluronic F-68. The cations in the corona solution may be calcium chloride, potassium chloride or sodium chloride.

Additionally, in all aspects of this embodiment in the core solution of the composition the polyanionic polymers may be present in a concentration of about 0.01 wt-% to about 0.5 wt-%. The monovalent or divalent salt may be present in a concentration up to about 3 wt-%. The cross-linking agent may be present in a concentration of about 0.01 wt-% to about 0.1 wt-%. The adenoviral polynucleotide conjugate may be present in a concentration of about 0.01 wt-% to about 0.1 wt-%.

Furthermore, in all aspects of this embodiment in the corona solution of the composition, the polycationic polymers may be present in a concentration of about 0.01 wt-% to about 1.0 wt-%. The cations may be present in a concentration of about 0.1 wt-% to about 3.0 wt-%. The targeting conjugate may be present in a concentration of about 0.01 wt-% to about 0.1 wt-%.

In a related aspect, in the core solution of the composition, the polyanionic polymers are sodium alginate and cellulose sulfate, the salt is sodium chloride, the polynucleotide is a gene and the crosslinking agent is dextran polyaldehyde. In a similar aspect, the core solution differs in that the polyanionic polymers are pentasodium tripolyphosphate and kappa (iota)-carrageenan. In these related aspects the corona solution may be as described supra.

In an additional aspect, in the core solution of the composition, the polyanionic polymers are sodium alginate and cellulose sulfate, the salt is sodium chloride or calcium chloride, the polynucleotide is a gene and the crosslinking agent is dextran polyaldehyde. In the corona solution the polycations are spermine hydrochloride, PMCG hydrochloride and F-68, the cation is calcium chloride and the targeting conjugate is a dextran-conjugated lectin or a dextran-conjugated glycan. In a similar aspect the core solution does not contain the dextran polyaldehyde. In another similar aspect the core solution does not comprise the dextran polyaldehyde and the corona solution does not comprise the dextran-conjugated lectin or the dextran-conjugated glycan.

In another aspect, in the core solution of the composition, the polyanionic polymers are pentasodium tripolyphosphate and kappa (iota)-carrageenan, the salt is sodium chloride, the polynucleotide is a gene and the crosslinking agent is dextran polyaldehyde. In the corona solution the polycations are chitosan glutamate and F-68, the cations are sodium chloride and/or calcium chloride and the targeting conjugate is a dextran-conjugated lectin or a dextran-conjugated glycan. In a similar aspect the core solution does not comprise the dextran polyaldehyde and the corona solution does not comprise sodium chloride. In another similar aspect the core solution does not comprise sodium chloride and the corona solution does not comprise a dextran-conjugated lectin or the dextran-conjugated glycan.

In another related embodiment of this invention, there is provided a composition comprising a water-based core solution which comprises sodium alginate; cellulose sulfate; an adenoviral gene construct; sodium chloride or calcium chloride; and, optionally, dextran polyaldehyde; and a water-based corona solution surrounding said core which comprises spermine hydrochloride; PMCG hydrochloride; pluronic F-68; calcium chloride; and, optionally, a dextran-conjugated lectin or a dextran-conjugated glycan; or a pharmaceutical composition thereof.

In all aspects of this embodiment in the core solution the concentrations of sodium alginate and cellulose sulfate is individually about 0.05 wt-%, the concentration of sodium chloride is about 2.0 wt-% or the concentration of calcium chloride is about 1.0 wt-%, the concentration of the adenoviral gene construct is about 0.1 wt-%, and the concentration of the optional dextran polyaldehyde is about 0.01 wt-% to about 0.1 wt-%. Additionally, in the corona solution the concentrations of spermine hydrochloride and PMCG hydrochloride are individually about 0.05 wt-%, the concentration of pluronic F-68 is about 1 wt-%, the concentration of calcium chloride is about 0.05 wt-%, and the concentration of the optional dextran-conjugated lectin or the dextran-conjugated glycan is about 0.01 wt-% to about 0.1 wt-%. Furthermore, the composition may be nanoparticulate, microcapsular or a polymer sheet.

In yet another related embodiment there is provided a composition comprising a water-based core solution which comprises pentasodium tripolyphosphate; kappa (iota)-carrageenan; an adenoviral gene construct; and, optionally, sodium chloride or dextran polyaldehyde or a combination thereof; and a water-based corona solution surrounding said core which comprises chitosan glutamate; pluronic F-68; calcium chloride; optionally, sodium chloride; and optionally, a dextran-conjugated lectin or a dextran-conjugated glycan; or a pharmaceutical composition thereof.

In all aspects of this embodiment in the core solution the concentrations of pentasodium tripolyphosphate and kappa (iota)-carrageenan are individually about 0.01 wt-%, the concentration of the optional sodium chloride is about 2.0 wt-%, the concentration of the adenoviral gene construct is about 0.01 wt-%, and the concentration of said optional dextran polyaldehyde is about 0.01 wt-% to about 0.1 wt-%. Additionally, in the corona solution the concentration of chitosan glutamate is about 0.05 wt-%, the concentration of pluronic F-68 is about 1 wt-%, the concentration of sodium chloride is about 1.0 wt-% and/or the concentration of calcium chloride is about 1.0 wt-%, and the concentration of the optional dextran-conjugated lectin or dextran-conjugated glycan is about 0.01 wt-% to about 0.1 wt-%. Furthermore, the composition may be nanoparticulate, microcapsular or a polymer sheet.

In still another embodiment of the present invention there is provided a method of delivering a polynucleotide or a drug to a human or a non-human animal to treat a pathophysiological state therein comprising administering any of the compositions described supra to the human or the non-human animal where the compositions contain a pharmacologically effective amount of the polynucleotide or of the drug. The components and concentrations thereof of the core solutions and the corona solutions are as described supra. The compositions may be nanoparticulate, microcapsular or a polymeric sheet.

As used herein, the term “drug” shall refer to a chemical entity of varying molecular size, both small and large, exhibiting a therapeutic effect in animals and humans.

As used herein, the term “gene” shall refer to any polynucleotide sequence representing a suitable protein product expressed in cells.

As used herein, the term “nanoparticle” shall refer to submicroscopic, i.e. less than 1 micrometer in size, solid object, essentially of regular or semi-regular shape.

As used herein, the term “microcapsule” shall refer to microscopic, i.e., a few micrometers in size to a few millimeters, solid object, essentially of regular spherical shape, exhibiting a liquid core and a semipermeable shell.

As used herein, the term “polymeric sheet” or “polymeric film” shall refer to a microscopic gelled solid object of slab geometry.

As used herein, the term “shell” or “corona” shall refer to an insoluble polymeric electrostatic complex composed of internal core polymer(s) and external bath polymer(s) molecularly bonded or gelled in a close proximity.

As used herein, the term “multiplicity of infection” shall refer to the amount of viral elements which are available for gene transfer.

As used herein, the term “adenoviral gene construct” shall refer to an engineered adenoviral vector possessing suitable introduced gene elements.

As used herein, the term “cations” shall refer to a combination of cations, such as, but not limited to, calcium chloride, potassium chloride or aluminum sulfate, and/or polycationic polymers.

As used herein, the term “polycation” shall refer to a polycationic polymer.

As used herein, the term “polyanion” shall refer to a polyanionic polymer.

As used herein, the term “core polymer” shall refer to an internal part of a nanoparticle, of a microcapsule or of a polymeric film.

As used herein, the term “light scattering” or “Tyndall effect” shall refer to light dispersion in many directions, resulting in a slightly milky suspension, visible by a human eye.

In the description of the present invention, the following abbreviations may be used: MOI, multiplicity of infection; AdV, adenoviral gene construct; SA-HV, high viscosity sodium alginate; CS, cellulose sulfate; k-carr, kappa carrageenan; LE-PE, low-esterified pectin (polygalacturonic acid); Chit, chitosan glutamate; PGA, polyglutamic acid; CMC, carboxymethylcellulose; ChS-6, chondroitin sulfate-6; ChS-4, chondroitin sulfate-4; F-68, Pluronic copolymer; GGT, γ-glutamyl transferase; DPA, dextran polyaldehyde; PVSA, polyvinylsulphonic acid; PVPA, polyvinyl phosphonic acid; PAA, polyacrylic acid; PVA, polyvinylamine; BSA, bovine serum albumin; 3PP, pentasodium tripolyphosphate; PMCG, poly(methylene-co-guanidine) hydrochloride; SH, spermine hydrochloride; PS, protamine sulfate; PEI, polyethyleneimine; PEI-eth, polyethyleneimine-ethoxylated; PEI-EM, polyethyleneimine, epichlorhydrin modified; Q-PA, quartenized polyamide; pDADMAC-co-acrylamide, polydiallyldimethyl ammonium chloride-co-acrylamide; PBS, phosphate-buffered saline; PEG, polyethylene glycol; PPG, polypropylene glycol; PEO, polyethylene oxide; HEC, hydroxyethyl cellulose; ACCP, SA/CS/CaCl₂/PMCG; and ACCSP, SA/CS/SP/CaCl₂/PMCG.

The present invention is based on a unique formulation method using multicomponent water-soluble polymers formed into nanoparticles, microcapsules or polymeric sheets. This preparation permits modification to a desirable size, provides adequate mechanical strength and exhibits exceptional permeability, surface characteristics and stability in the presence of salt or sera. Many polymeric combinations of water-soluble polyanions and polycations may be suitable for generation of, inter alia, nanoparticles. Such combinations may result in a precipitated complex, which is acceptable as long as it remains insoluble after its formation or an electrostatic insoluble complex. Soluble complexes are ineffective as no particle formation occurs. Both precipitated and electrostatic complexes are desirable for nanoparticle formation. It is also contemplated that polyanions and polycations interact to form microcapsules and polymeric sheets or films that have a different geometry and size.

The criterium for selection was formation of submicroscopic particles as demonstrated by the Tyndall effect. The nanoparticle size and charge can be measured by means of a Malvern ZetaMaster (Malvern, UK). Individual polymers were tested for biocompatibility using an in vitro culture system with rat insulinoma cells (RIN 1046-38 cells, American Type Culture Collection, Rockville, Md.). Some of the possible combinations are listed in TABLE I. The anionic components do not include mono- or divalent salts such as sodium chloride, calcium chloride or sodium sulfate which may be included with the polyanions.

TABLE I
Multicomponent particulate systems

| Anionic components | Cationic components |
|---|---|
| SA-HV/3PP | Chit/calcium chloride |
| SA-HV/LE-pectin | BSA/calcium chloride |
| LE-pectin | PEI/calcium chloride |
| ChS-4/SA-HV | Gelatin A/calcium chloride |
| LE-pectin/SA-HV | Gelatin A/calcium chloride |
| Acacia/SA-HV | Gelatin A/calcium chloride |
| κ-carr/SA-HV | BSA/calcium chloride/potassium chloride |
| CS/SA-HV | Chit/calcium chloride |
| Sodium sulfate/SA-HV | Chit/calcium chloride |
| Gelatin B/SA-HV | Chit/calcium chloride |
| ChS-6/SA-HV | Chit/calcium chloride |
| ChS-4/SA-HV | Chit/calcium chloride |
| Gellan/SA-HV | PLL/calcium chloride |
| LE-pectin/SA-HV | Q-PA/calcium chloride |
| SA-HV/CS | Chit/calcium chloride |
| SA-HV/PGA | Chit/calcium chloride |
| CS/PGA | Chit/calcium chloride |
| Xanthan/gellan | PLL/calcium chloride |
| Xanthan/CS | PEI-eth/calcium chloride |
| Xanthan/k-carr | PEI-eth/calcium chloride/potassium chloride |
| Xanthan/gellan | PEI-eth/calcium chloride |
| Xanthan/CS | PEI-EM/calcium chloride |
| Xanthan/CMC/ | pDADMAC-co-acrylamide/aluminum sulfate |
| CS/SA-HV | PVA/calcium chloride |
| CS/CMC | PVA/calcium chloride/aluminum sulfate |
| CS/gellan | PVA/calcium chloride |
| CMC/gellan | PVA/ aluminum sulfate/calcium chloride |
| CS/CMC | Q-PA/calcium chloride |
| CS/xanthan | Q-PA/calcium chloride |
| CS/κ-carr | Q-PA/calcium chloride |
| CS/gellan | Q-PA/calcium chloride |
| CMC/xanthan | Q-PA/calcium chloride |
| CMC/κ-carr | Q-PA/calcium chloride |
| CMC/gellan | Q-PA/calcium chloride |
| Xanthan/κ-carr | Q-PA/calcium chloride |
| Xanthan/gellan | Q-PA/calcium chloride |
| CS/CMC | Polybrene |
| CS/xanthan | Polybrene |
| CS/κ-carr | Polybrene |
| CS/gellan | Polybrene |
| CMC/xanthan | Polybrene |
| CMC/κ-carr | Polybrene |
| CMC/gellan | Polybrene |
| Xanthan/κ-carr | Polybrene |
| Xanthan/gellan | Polybrene |
| PVPA/SA-HV | Chit/calcium chloride |
| PVSA/SA-HV | Chit/calcium chloride |
| PVPA/CS | Chit/calcium chloride |
| PVSA/CS | Chit/calcium chloride |
| SA-HV/3PP | PVA/calcium chloride |
| CS/3PP | PVA/calcium chloride |
| CMC/3PP | PVA/calcium chloride |
| CMC/3PP | PVA/calcium chloride/aluminum sulfate |
| Gellan/3PP | PVA/calcium chloride |
| Xanthan/SA-HV | PLL/SP |
| SA-HV/gellan | SH/PMCG |
| SA-HV/CS | SH/PMCG |
| SA-HV/gellan | PH/PMCG |
| SA-HV/CS | PH/PMCG |
| SA-HV/gellan | Polybrene/PMCG |
| SA-HV/CS | Polybrene/PMCG |
| κ-carr | PS/calcium chloride/potassium chloride |
| κ-carr/SA-HV | PS/calcium chloride/potassium chloride |
| κ-carr | SP/calcium chloride/potassium chloride |
| κ-carr/SA-HV | SP/calcium chloride/potassium chloride |
| κ-carr | Polybrene/calcium chloride/potassium chloride |
| κ-carr/SA-HV | Polybrene/calcium chloride/potassium chloride |
| κ-carr/heparin | PS/potassium chloride |
| κ-carr/heparin | Polybrene/potassium chloride |
| κ-carr/heparin | SH/potassium chloride |
| CS/heparin | PS/calcium chloride/potassium chloride |
| CS/heparin | Polybrene/calcium chloride/potassium chloride |
| CS/heparin | SH/calcium chloride/potassium chloride |
| PVSA/SA-HV | Chit/calcium chloride |
| κ-carr/gellan | PVA/calcium chloride |
| SA-HV/gellan | PVA/calcium chloride |
| PAA/SA-HV | Chit/calcium chloride |
| PAA/CS | Chit/calcium chloride |
| PAA/gellan | Chit/calcium chloride |
| PAA/κ-carr | Chit/calcium chloride |
| 3PP/κ-carr | Chit/calcium chloride |
| SA/CS | calcium chloride/PMCG |
| SA/CS | calcium chloride/SH/PMGC |

The present invention is directed to a composition of matter comprising various polyanionic/polycationic polymer compositions incorporating a polynucleotide or nucleic acid such as gene constructs based on AdV or other drug. Among useful polyanions for making polymeric films, microcapsules and nanoparticles are sodium algenate, kappa carrageenan, pentasodium tripolyphosphate, low-esterified pectin, polyglutamic acid, cellulose sulfate and chondroitin sulfate. Possible polycations include, polyvinylamine, spermine hydrochloride, protamine sulfate, polyethyleneimine, polyethyleneimine-ethoxylated, polyethyleneimine-epichlorhydrin modified, quarternized polyamide, polydiallyldimethyl ammonium chloride-co-acrylamide, and chitosan glutamate, among others.

Preferably, the nanoparticles are synthesized from the polyanions high viscosity sodium alginate and cellulose sulfate and the calcium chloride and poly(methylene-co-guanidine) hydrochloride (PMCG) (polycation) and spermine hydrochloride. It is also preferred that the polyanionic core comprises a monovalent or divalent salt such as sodium chloride, calcium chloride, or sodium sulfate. Gene constructs could be represented by any suitable therapeutic gene.

The increased stability of the particles results in, inter alia, increased entrapment efficiency for a more efficacious delivery of a biomolecule contained within the core of the particle. A particularly usable combination is one of a polynucleotide, such as an adenoviral gene construct, or a drug and SA-HV/CS as the polyanion or a CaCl₂/SP/PMCG complex as the polycation.

Particles may be made in a stirred reactor. The reactor is filled with a cationic solution. An anionic core solution is mixed into the cationic corona or shell solution residing in the reactor or receiving bath. Typically, 1-2 ml of anionic solution is mixed into 20 ml of corona solution in a batch mode, instantly resulting in an insoluble nonstoichiometric complex with an excess of cationic charge on the particle periphery. Usually, 1-2 hours is sufficient for particle reaction and maturation. The nanoparticle size and charge was evaluated in the reaction mixture by centrifugation at 15,000 g. A monovalent or bivalent salt, such as sodium chloride, calcium chloride, or sodium sulfate may be included in the anionic solution.

Resulting nanoparticles consist of a dense anionic polymeric core matrix optionally comprising the monovalent or bivalent salt. The nanoparticles are stable in the presence of salts and sera. Even when the nanoparticles are made in the presence of salts, the stability of the polyelectrolyte complex is extremely high. In addition, the entrapment efficiency is increased many times.

A polynucleotide or drug can be dispersed or dissolved in the anionic core. These compositions may be included with the anionic solution during particle production. Particularly, it is contemplated that an adenoviral gene construct is loaded into the polyanionic core. Loading of the polyanionic core may occur during particle formation.

Additionally, release of the AdV gene construct or other drug may be controlled by incorporating a crosslinking polymer, e.g., dextran polyaldehyde in the anionic core during particle formation. The crosslinking polymer provides mechanical strength for the nanoparticle and permeability control for release of the AdV gene construct. Furthermore, it is contemplated that the nanoparticles may be targeted to a cell of interest by entrapping a conjugate comprising lectin or glycan within the corona or shell of the nanoparticle during production.

The individual components of the core polyanionic solution of polymers include concentrations of about 0.01 wt-% to about 0.5 wt-%. In a more preferred composition, each component of the polyanions is at a concentration of about 0.05 wt-% to about 0.2 wt-%. The mono- or divalent salts may be at a concentration of 0 wt-% to about 3.0 wt-% in the polyanionic core. The individual components of the corona cationic solution are at a concentration of about 0.01 wt-% to about 1.0 wt-%. In a more preferred composition, the corona polycations are at about 0.05 wt-% to about 1.0 wt-% and calcium chloride at about 0.05 wt-% to about 0.1 wt-% or potassium chloride at about 0.05 wt-% to about 0.2 wt-% in case carrageenans are used as anionic polymers.

A crosslinking agent, such as dextran polyaldehyde, may comprise the anionic core in concentrations up to about 0.1 wt-%. As the PDA concentration increases, the cumulative delivery time increases. Release rate of the AdV gene construct or drug may vary from about 3%/day to about 20%/day which yields a delivery time of about 30 days to about 10 days or possibly less.

The particles described herein are useful for drug delivery. The present invention provides a multicomponent particle formed by polyelectrolyte complexation. In case the drug or targeted biological substance is polyelectrolyte by virtue of its nature, such components become an integral part of the particle core. Therefore it is contemplated that a pharmaceutical composition may be prepared using a drug encapsulated in the nanoparticulate delivery vehicle of the present invention. In such a case, the pharmaceutical composition may comprise a drug, e.g., an anti-vascularization agent, and a biologically acceptable matrix. A person having ordinary skill in this art readily would be able to determine, without undue experimentation, the appropriate concentrations of such typical biotechnology products, matrix composition and routes of administration of the vehicle of the present invention.

It is further contemplated that the nanoparticles comprising an AdV gene construct or other drug are used to treat or provide other therapeutic benefit to a pathophysiological state in an animal or mammal. Such pathophysiological state may include cancers such as squamous cancers, head and neck cancer or lymphoid-derived metastases or may be delivered to dendritic cells or to islet cells to provide treatment or a therapeutic benefit to a pathophsiological state involving these cells. It is further contemplated that entrapping targeting agents within the corona of the nanoparticle specifically increases efficacy of the nanoparticles in targeting specific cells.

The following examples are given for the purpose of illustrating various embodiments of the invention and are not meant to limit the present invention in any fashion.

### EXAMPLE 1

Generation of Nanoparticles for In Vitro Gene Transfer

These particles were generated using a droplet-forming polyanionic solution composed of 0.05 wt-% SA-HV, 0.05 wt-% CS and 0.008 wt-% pCEPluc plasmid in water, and corona-forming polycationic solution composed of 0.05 wt-% SH, 0.065 wt-% PMCG, 0.05 wt-% CaCl₂ and 1.0 wt-% F-68 in water. The latter solution was used as a plasmid condensing agent. pCEPluc is plasmid with a CEP promoter, covalently linked to a luciferase gene as a reporter gene. This plasmid was expressed in a bacterium, grown in a culture and isolated in-house. The ratio of droplet- to corona-forming reactants was 1:10.

For particle generation, a special glass double-nozzle atomizer was used. The droplet-forming solution was applied in the internal nozzle, while the air was used to strip particles off the internal nozzle and atomize them into submicron-range size using an internal nozzle. The droplets were then collected in the corona-forming solution. Such device was used because the DNA molecule is sensitive to sonication and can be substantially damaged. The particles were separated by centrifugation and washed. Their size and charge were 190 nm and +24.0 mV, respectively. These particles exhibited an expression of luciferase enzyme in several in vitro cell culture lines.

### EXAMPLE 2

Use of Nanoparticles for In Vivo Delivery of Plasmid DNA

These particles were generated using a droplet-forming anionic solution containing 0.025 wt-% pCEPluc plasmid in water, and corona-forming cationic solution composed of 0.05 wt-% Tetronic 904 (BASF) in water. The ratio of droplet- to corona-forming reactants was 1:1. Two reactants were simply mixed together with the polyanion added to the polycation to form nanoparticles. Their size and charge were 190 nm and +24.0 mV, respectively. The particles were resuspended in isotonic 5 wt-% glucose solution and injected intradermally into 5 experimental animals (see Example 9), 0.1 ml per site. Six sites have been applied per animal. Each animal had 2 negative controls (5 wt-% glucose) and two positive controls (5 wt-% glucose, 0.025 wt-% Lipofectamine (Gibco, Gaithersburg, Md.), 0.025 wt-% pCEPluc plasmid). Animals were harvested after 24 hours by means of 8 mm skin punch. Gene expression was measured by assaying for luciferase activity in minced and permeabilized cell extracts, using a luminometer and data were normalized per protein content. The commercial luciferase assay kit (Sigma) was used. In another set of experiments, empty nanoparticles were used as another negative control with values of RLU/protein close to the negative control.

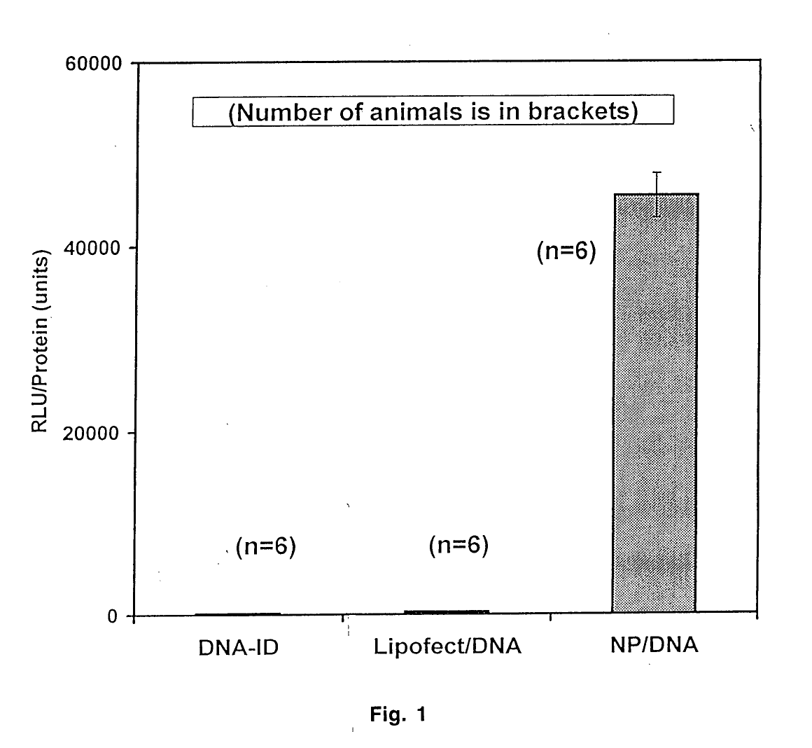

Results are presented in FIG. 1. The values presented as a bar height represent the average (n=number of sites)+/−SD. These results clearly show that the formulated plasmid can achieve quite efficient gene transfection, many times over the baseline (controls) (about 400 times over the negative control). Similar results were obtained for polyanionic solution containing 0.025 wt-% pCEPluc and 0.005 wt-% SA-HV and polycationic solution containing 0.05 wt-% Tetronic 904 and 0.005 wt-% CaCl2 in water. Some other detergents of the Pluronic and Tetronic series (BASF) worked equally well.

### EXAMPLE 3

Nanoparticles Have Increased Entrapment Efficiency and Stability in the Presence of Salt and Sera

An increased amount of a monovalent or bivalent salt, e.g. sodium chloride, calcium chloride, or sodium sulfate may be added to the anionic solution prior to forming the polyelectrolyte complexes as described.

The stability of two nanoparticles prepared in the presence of sodium chloride and their entrapment efficiency were monitored. ACCSP nanoparticles were generated using a droplet-forming polyanionic solution composed of core solution containing 0.05% sodium alginate (Kelco), 0.05% cellulose sulfate (Janssen Chimica) and corona solution containing 0.05% calcium chloride (Sigma) and 0.075% poly(methylene-co-guanidine) chloride (Scientific Polymer Products), 0.05% Spermine hydrochloride and 1% F-68 (Pluronic). 2 milliliters of core solution is collected in 50 milliliters of corona solution. In addition, the core solution contained 0 wt % to about 2.0 wt % sodium chloride.

Table II shows the entrapment efficiency of ACCSP, a quaternary complex, prepared in presence of sodium chloride solutions ranging from about 0 to 3 wt-%. Stability was tested over a period of several months in 0.9% NaCl solution and sera. The nanoparticles were measured daily by a Malvern ZetaSizer instrument over a period of several weeks. For stability in sera, nanoparticles were resuspended in mice serum and size monitored over the required period of time. While the size in 0.9 wt-% sodium chloride increased slowly over time, i.e., doubled in 2 weeks, the size of the nanoparticles prepared in the presence of a core sodium chloride concentration of about 1-3 wt-% remained stable for long period of time. The size of nanoparticles remained the same as initially measured, i.e., 220 nm+/−25 nm. In both tests in 0.9 wt-% NaCl and in serum, the amount of precipitate did not change with time, that is the amount of mass produced was stable.

TABLE II
Stability of ACCSP prepared in presence of sodium
chloride and effect on entrapment efficiency

| Amount of NaCl in | | |
|---|---|---|
| Core solution (%) | Entrapment Efficiency % | Stability |
| 0 | 4.1 | extremely high |
| 1.00 | 13.1 | extremely high |
| 1.50 | 19.5 | extremely high |
| 2.00 | 28.3 | extremely high |
| 2.50 | 33.4 | extremely high |
| 3.00 | 38.5 | extremely high |

Similar data were obtained for calcium chloride and sodium sulfate solutions in the range of 0-3 wt-%. Similar results as in Table VI were obtained for protein-loaded nanoparticles (data not shown). Intravenous application of nanoparticles. into mice (tail vein) also corroborates the stability of these nanoparticles and their nonaggregation in vivo.

### EXAMPLE 4

Adenoviral Gene-Loaded Nanoparticle Production Process

The polymers used to produce the nanoparticles are high viscosity sodium alginate (SA-HV) (Kelco/Merck, San Diego, Calif.) with an average molecular weight of 46,000, cellulose sulfate, sodium salt (CS) (Janssen Chimica, Geel, Belgium) with an average molecular weight of 1,200,000; poly(methylene-co-guanidine) hydrochloride (PMCG) (Scientific Polymer Products, Inc., Ontario, N.Y.) with an average molecular weight of 5,000, and spermine hydrochloride (SH) (Sigma) with a molecular weight of 348.2. Pluronic P-68 (Sigma) with an average molecular weight of 5,400 is a water-soluble nonionic block polymer composed of polyoxyethylene and polyoxypropylene segments.

Particles were generated using a droplet-forming polyanionic solution composed of 0.05 wt-% HV sodium alignate (SA-HV), 0.05 wt-% CS in water, 0.01-0.1 wt-% adenoviral gene construct and also containing 2 wt-% NaCl (Sigma), and a corona-forming polycationic solution composed of 0.05 wt-% SH, 0.05 wt-% PMCG hydrochloride, 0.05 wt-% calcium chloride, and 1 wt-% F-68 in water. The particles were formed instantly via mixing 2 ml of the core solution with 20 mls of the corona solution and were allowed to react for 1 hour under stirring. Furthermore, these nanoparticles were also prepared in the presence of 0-2 wt-% NaCl or 0-2% calcium chloride added into the polyanionic droplet-forming solution.

The encapsulation efficiency was 40% based on DNA measurements. The nanoparticle size and charge was evaluated in the reaction mixture by centrifugation at 15,000 g. The average size was 230 nm and the average charge +15.2 mV. The particles were resuspended with different buffers, e.g., neutral pH 7, pH 1.85 and pH 8, and plasmid release was measured by a colorimetric method. The product is stable in water, neutral buffers, in 0.9 wt-% saline and in animal sera.

### EXAMPLE 5

Adenoviral Gene-Loaded Nanoparticle 1 and Controlled Release

These particles were generated using the same solutions as Example 4, except the droplet forming solution contained additional polymer, PDA and 1 wt-% calcium chloride instead of sodium chloride. PDA is dextran polyaldehyde (CarboMer, Westborough, Mass.) with an average molecular weight of 40,000. The particles were instantaneously formed, allowed to react for 1-hour and their size and charge evaluated in the reaction mixture. The average size was 250 nm and the average charge was 15.5 mV. The particles were separated by centrifugation and incubated for 30 min. in a HEPES buffer at pH 8.0 to perform the crosslinking reaction between the polymer constituent and PDA. The PDA concentrations were: 0 (no crosslinking), 0.01, 0.03 and 0.06 wt-%.

The Schiff-base product between the anionic groups of the core polymers and the aldehyde group of PDA allowed an adjustment of release via increase of the polymer chain entanglement. This way the release rate was adjusted to any value between 10 and 20%/day to 3%/day and 10%/day which resulted in approximately 30 to 10 days of cumulative delivery time. The tracer quantity was assayed via fluorescence. The permeability was assessed via an efflux method (12).

### EXAMPLE 6

Adenoviral Gene-Loaded Nanoparticle 2 and Controlled Release

A nanoparticle delivery vehicle different from that in Example 5 was assembled. It contained core-loaded adenoviral construct at different loadings. To allow for controlled release of the core-loaded gene construct, the release rate was adjusted by means of PDA crosslinking. A slow-release of the core adenoviral construct is important for achieving sustained gene delivery. Several concentrations were applied in order to allow for a slow-delivery over a 10 days period, i.e., the total release time is adjusted to 10 days.

Particles were generated using droplet-forming polyanionic solution composed of 0.1 wt-% pentasodium tripolyphosphate (3PP, anhydrous; Sigma, St., Louis, Mo.), 0.1 wt-% κ-carrageenan (X-52; Sanofi Bio-Industries, Waukesha, Wis.), and 0.01-0.1 wt-% adenoviral gene construct and corona-forming solution composed of 0.05 wt-% chitosan glutamate (Pronova Biopolymer, Drammen, Norway), 0.1 wt-% calcium chloride (Sigma), 1 wt-% sodium chloride, and 1 wt-% Pluronic F-68 (Sigma).

### EXAMPLE 7

Biocompatibility Test of Gene-Loaded Nanoparticles

Nanoparticles loaded with 0.1 wt-% /batch of adenoviral gene (AdV) construct were prepared as described in Example 5. Nanoparticles prepared in the absence of AdV were produced as a control. Nanoparticles and, separately, controls were injected subcutaneously and intraperitoneally into Sprague-Dawley rats at 0.2 ml each and evaluated at days 8, 48 and 96. Visual observation, backed by histology (inflammatory reactions, degree of fibrosis and development of granulation tissue with capillaries) revealed that the product is biocompatible.

Biocompatibility of the empty nanoparticle, i.e., no gene inserted, prepared as above was determined in the subcutaneous and intraperitoneal sites in rats. Histology and histochemistry of all implants included standard techniques [13-14]. No adverse reactions were noted.

### EXAMPLE 8

Nanoparticulate Gene Transfer Into Cancer Cells

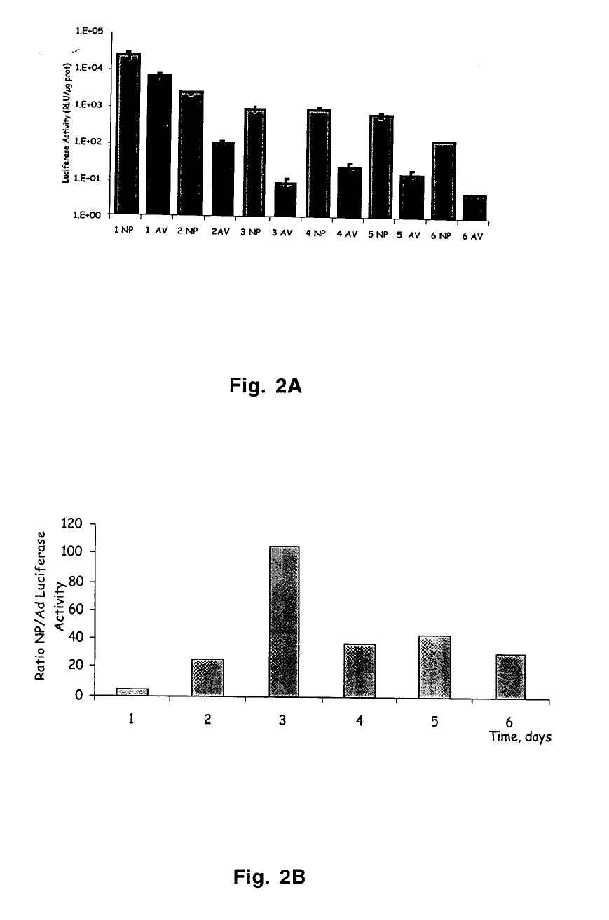

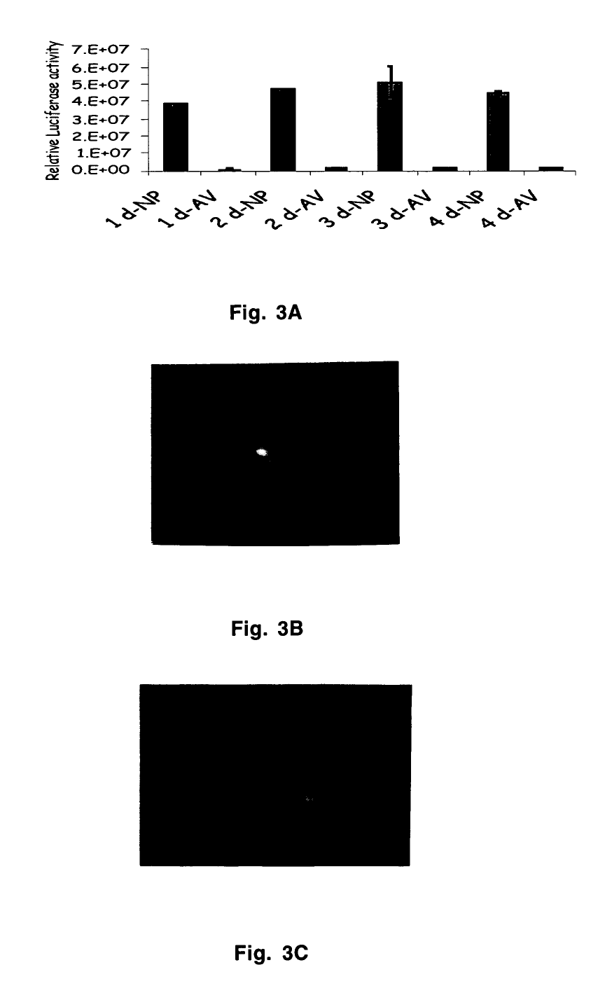

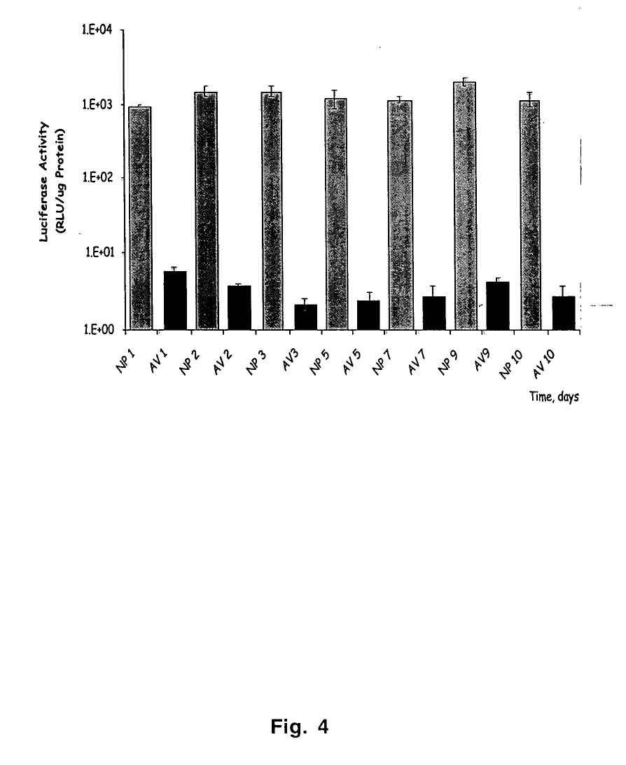

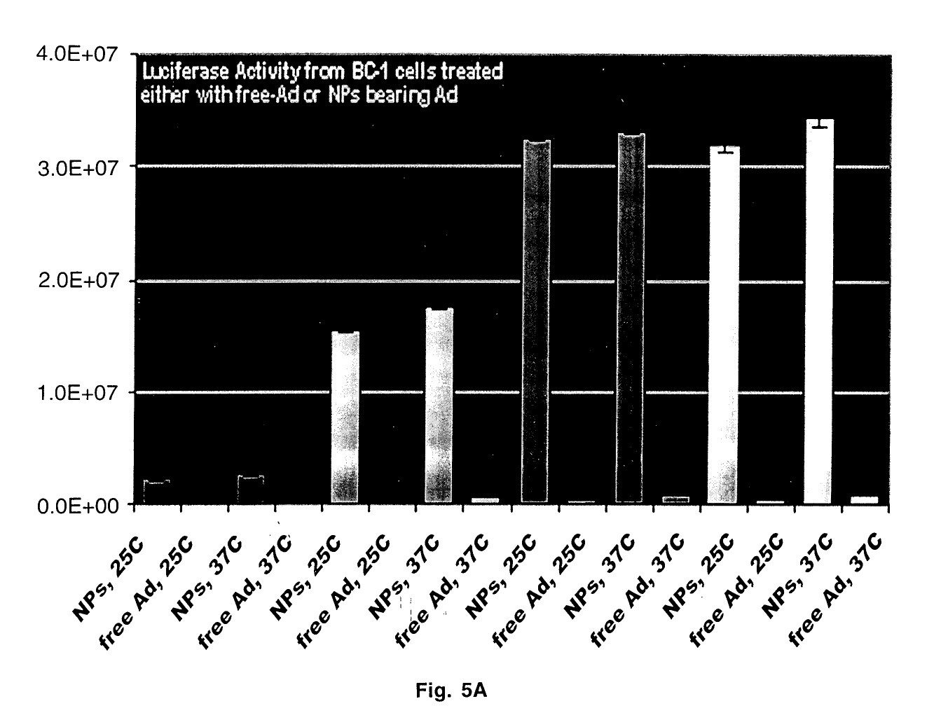

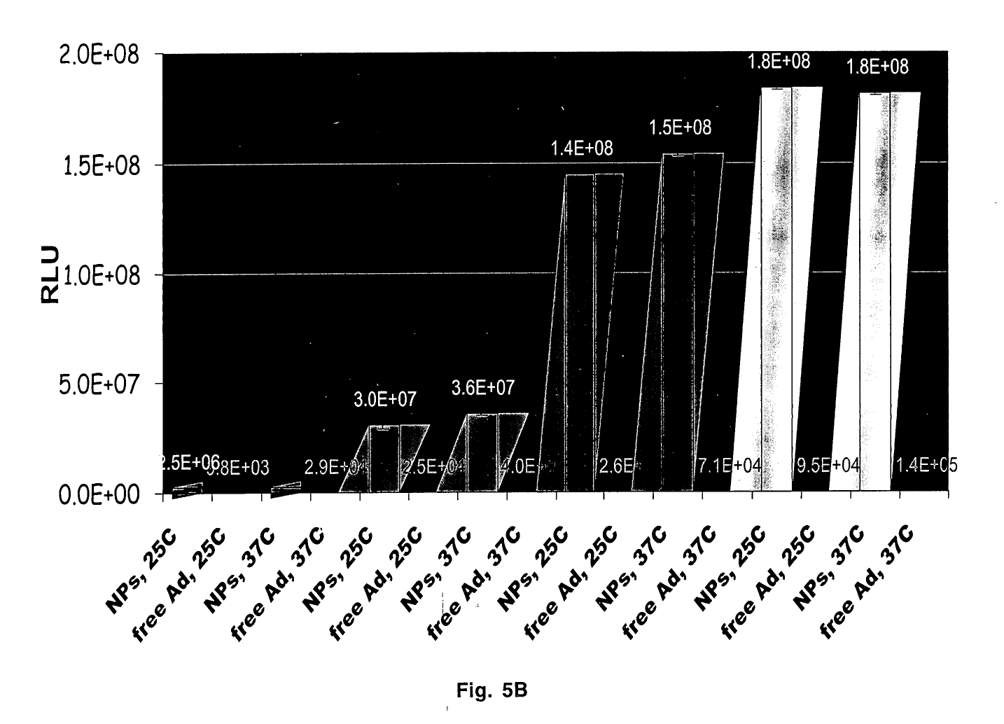

The cell lines, which have been tested for gene transfer using nanoparticular technology described herein, were UMR-106, a clonal derivative of a transplantable rat osteosarcoma that had been induced by injection of radiophosphorous ³²P (FIGS. 2A-2B), CT26 (FIGS. 3A-3C), HT1080 (FIG. 4), and BC-1 and BC-BL-1, both lymphomas (FIGS. 5A-5B). The cells were treated either with free adenovirus or nanoparticles containing the adenovirus encoding luciferase for 2 hours under gentle agitation at room temperature and at 37° C. (MOI=5, tested by plaque assay using 293 NIH cells). Then cells were washed once with PBS in order to remove the unbound Adenovirus and new growth medium was added. Luciferase activity was measured every 24 hours up to 4 days.

### EXAMPLE 9

Nanoparticulate Gene Transfer Into Islet Cells

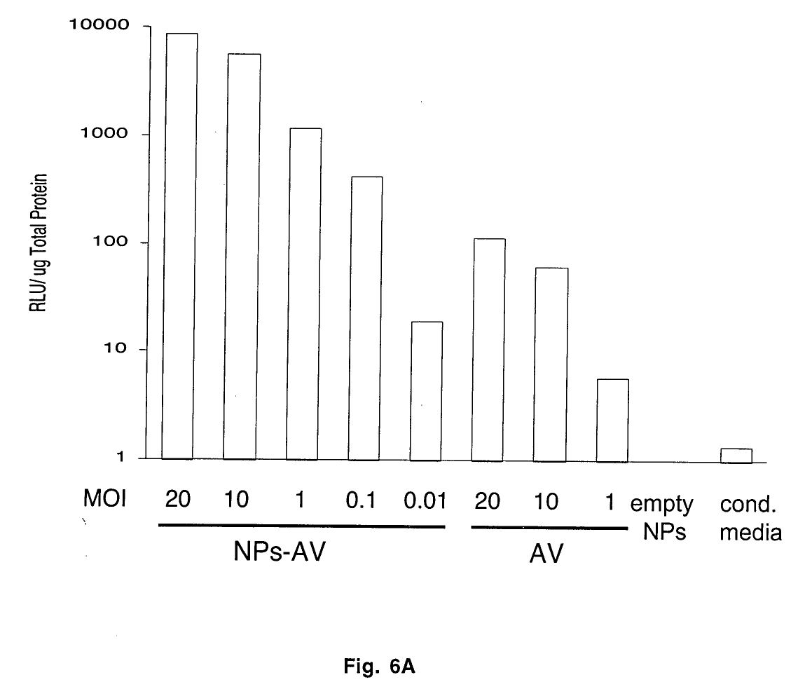

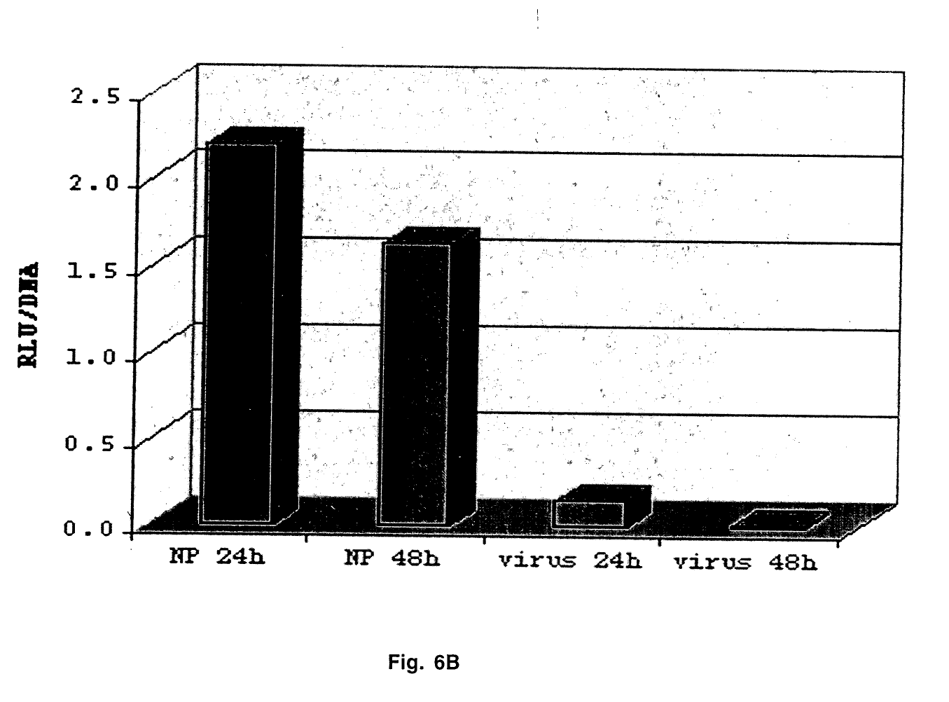

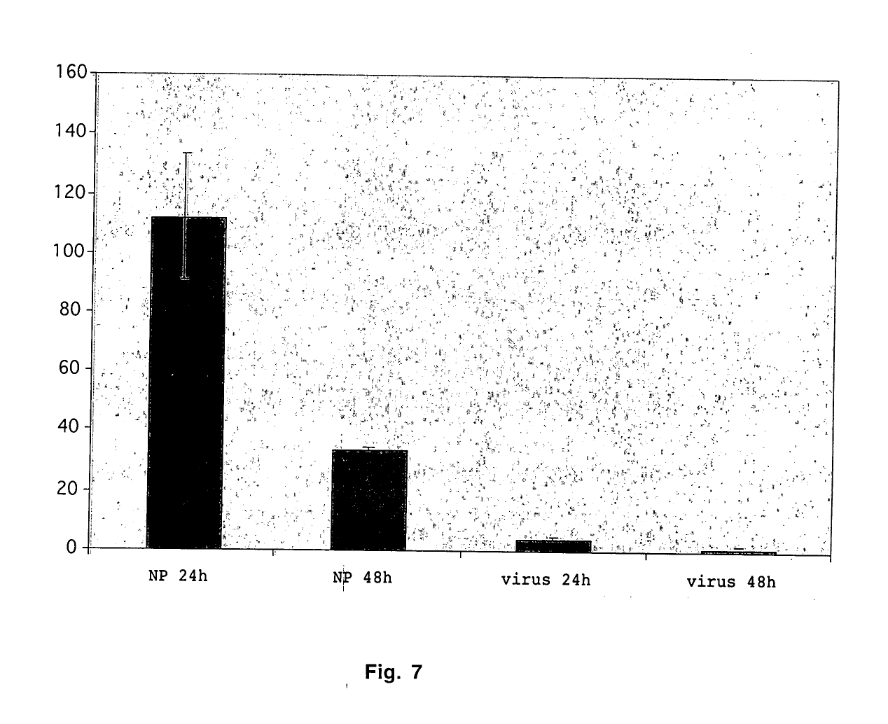

Islet cells were infected by adding either nanoparticle suspension containing 1.8×10⁵ pfu in 10 μl of Adenovirus-Luciferase or 1×10⁶ pfu of stock solution Adenoviruse-Luciferase in 1 μl per well. The infection was carried out overnight using the same medium used to grow the cells, i.e., 200 μl RPMI 1640/well. The following day islets were washed twice with PBS and 500 μl of growth medium was added per well. At indicated time-points, 24 and 48 hours after the infections were performed, islets were washed with PBS, lysed using reporter lysis buffer (RLB by Promega Corp. Madison, Wis., USA), harvested and analyzed for gene expression. To measure the luciferase activity, the cells were freeze-thawed once, spun down and analyzed with a Luciferase Assay System Kit (Promega Cop. Madison, Wis., USA) according to the manufacturer's instructions (Technical Bulletin No. 281), using a 20-μl of aliquots of cells lysate with 100 μl luciferase assay buffer. The light emitted during 10 seconds was measured by a luminometer (Pharmigen, USA) set for a single photon counting. The total protein concentration (FIG. 6A) was determined by the bicinchoninic acid protein assay from Pierce Chemical Co. (Rockford, Ill.) with a bovine albumin as a standard.

For the DNA assay (FIG. 6B), 20 μl of each lysate cells were added into replicate weels of 96-well microplate (Costar). Picogreen reagent was diluted 360-fold in TE buffer, and 180 μl of diluted Picogreen reagent was added to each well. The microplate was stored in the dark for 4-5 minutes and then read on a fluorescent microplate reader (Cytofluor) with excitation and emission settings of 485 and 530 nm respectively. Picogreen signals from bacteriophage λ DNA samples were used to construct a linear six-point standard curve.

### EXAMPLE 10

Nanoparticulate Gene Transfer Into Dendritic Cells

Dendritic cells (DC) were generated on a weekly basis using peripheral blood obtained from healthy adult with previous informed consent. PMBCs were separated using Ficoll gradient. PMBCs were plated in culture dishes in 10% FBS RPMI-1640 medium supplemented with IL-4 and GM-CSF at 37° C. in a humidified atmosphere flushed with 5% CO₂. The transduction of dendritic cells was performed at day 4 by adding into each well, which contained 300,000 cells, the indicated amounts of Ad-NPs or free Ad-luc-IRES-GFP, expressed as MOI, directly to dendritic cell cultures and incubated for 6 hours. After washing dendritic cells were replated with fresh 10% FBS RPMI-1640 medium supplemented with IL-4 and GM-CSF. Dendritic cells were harvested at day 7, i.e., 60 hours post transfections with NPs-AV or infection with AV, lysed with 1× Reporter Lysis Buffer (RLB, Promega) and assayed for firefly luciferase. Values were normalized against total protein quantification by BCA assay (Pierce).

### EXAMPLE 11

Synthesis of a Dextran/A-Tetra Conjugate

A tetrasaccharide (A-tetra) specific for Galectin-3 was obtained from Dextra-Labs, UK. Its composition is as follows: GalNAc alpha1-3Gal beta1-4Glc (-2 Fuc alpha1) (15). The preparation of Dex/A-tetra conjugate was carried out according to the following procedure. Dextran (Molecular weight 4.2×10⁴, 1000 mg, 4.5 nmol in sugar unit; Sigma) was dissolved in dimethyl sulfoxide (DMSO, Sigma). 4-Nitrophenylchloroformate (650 mg, 3.2 mmol, Sigma) and 4-(dimethylamino)pyridine (DMAP, 350 mg, 2.8 mmol, Sigma) were added to the ice-cooled solution. The reaction mixture was stirred at 0° C. for 4 h and then reprecipitated by acetone/diethyl ether/ethanol (1:1:2, v:v:v) to give Dextran-activated ester.

The activated ester was dissolved in DMSO, and then A-tetra was added to the solution. The mixture was stirred at room temperature for 36 h. After evaporation, the residue was dissolved in DMF and subjected to gel-filtration chromatography (Sephadex LH-20; column, o.d. 40×550 mm; eluent, DMF) to give Dex/A-tetra conjugate. The degree of introduction of Gal units per sugar unit was estimated to be 2.9 mol % from the N:C ratio of the elemental analysis. The yield was 520 mg.

A control conjugate having no galactose residues was also synthesized; saccharose was used instead. These conjugates were used for the investigations of interactions with lectin (Galectin-3). The interactions of dextran derivatives with Galectin-3 lectin were evaluated by calorimetric titration (15). Results of the interactions between the lectin and dextran derivatives showed high apparent affinity constants for active conjugate.

### EXAMPLE 12

Nanoparticle Targeting Using Dex/A-Tetra Conjugate

The nanoparticle delivery vehicle similar to that in Example 5 was assembled. It contained core-loaded adenoviral construct and corona loaded Dex/Tetra-A conjugate at different loadings. The processes of targeting can be controlled by the absolute amounts of Dex/A-Tetra corona-loaded material in the range of about 0.01 to 0.1 wt-%. Nanoparticles exhibited a high affinity to a squamous tumor cell tissue section and a head and neck cancer cell line, as detected histochemically and/or by means of fluorescence by using a fluorescing polymer core-entrapped in the nanoparticles to simplify the observation (data not shown) (16). In a similar way, we also tested a targeting based on lectin, instead of glycan. In this case, SNA (Sambus nigra agglutinin, lectin, Vector Laboratories, Burlingame, Calif.) was incorporated into the nanoparticle corona by entrapment with a goal of targeting it to appropriate cell-based receptor, i.e., sugar-based, on the cell's periphery of the gastrointestinal tract, e.g., CaCo cells.

The following references are cited herein:

1. Desay, P. B., Microencapsulation of drugs by pan and air suspension technique. Crit. Rev. Therapeut. Drug Carrier Syst., 5: 99-139 (1988).

2. Berthold, A., Cremer, K., Kreuter, J. Preparation and characterization of chitosan microspheres as drug carrier for prednisolone sodium phosphate as model anti-inflammatory drugs. J. Controlled Release 39: 17-25 (1996).

3. Watts, P. J., Davies, H. C., Melia, C. D. Microencapsulation using emulsification/solvent evaporation: An overview of techniques and applications. Crit. Rev. Therapeut. Drug Carrier Syst. 7: 235-159 (1990).

4. Cowsar, D. R., Tice, T. R., Gilley, R. M., English, J. P. Poly(lactide-co-glycolide) microcapsules for controlled release of steroids. Methods Enzymol. 112: 101-116 (1985).

5. Genta, I., Pavanetto, F., Conti, B., Ginnoledi, P., Conte, U. Spray-drying for the preparation of chitosan microspheres. Proc. Int. Symp. Controlled Release Mater. 21: 616-617 (1994).

6. Sternberg et al., New structures in complex formation between DNA and cationic liposomes visualized by freeze-fracture electron microscopy. FEBS Letters 356: 361-366 (1994).

7. Milson, R. W. and Bloomfield, V. A. Counterion-induced condensation of DNA. A light-scattering study. Biochemistry 18: 2192-2196 (1979).

8. R. Langer, Drug delivery and targeting. Nature 392: 5-10 (1998).

9. M. Nishikawa and L. Huang: Nonviral vectors in the new millenium: delivery barriers in gene transfer, Human Gene Therapy 20: 861-870 (2001).

10. L. Vu. Tryon-Le, Scott M. Walsh, Erik Schweibert, Hai-Quan Mao, William B. Guggino, J. Thomas August and Kam W. Leong: Gene transfer by DNA-gelatin nanospheres, Archive of Biochemistry and Biophysics 361 47-56 (1999).

11. A. Prokop, E. Kozlov, G. Carlesso, J. M. Davidson: Hydrogel-based colloidal polymeric system for protein and drug delivery: Physical and chemical characterization, permeability control and applications. Advance Polymer Sci. 160: 119-173 (2002).

12. Prokop, et al., Water-soluble polymers for immunoisolation. ][ Evaluation of multicomponent microencapsulation systems. Advances in Polymer Science, 136: 52-73 (1998).

13. Sewell, W. R., Wiland, J. and Craver, B. N. New method of comparing sutures of bovine catgut in three species, Surgery in Gynecology and Obstetric 100: 483 (1955).

14. Spector, M., and Lalor, P. A. In vivo assessment of tissue compatibility. In: Biomaterials Science, An Introduction to Materials in Medicine, Ratner, B D, Hoffman A S, Schoen F J, and Lemons J E, eds., Academic Press, pp. 220-228 (1996).

15. Bachhawat-Sikder, Thomas and Surolia. Thermodynamic analysis of the binding of galactose and poly-N-acetyllactosamine derivatives to human galectin-3. FEBS Letters 500: 75-79 (2001).

16. Plzak, Smetana, Krdlickova, Kodet, Holikova, Liu, Dvorankova, Kaltner, Betka and Gabius Expression of galectin-3-reactive ligands in squamous cancer and normal epithelial cells as marker of differentiation. International Journal of Oncology 19: 59-64 (2001).

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A composition comprising:
a water-based core solution comprising:
polyanionic polymers; and
an adenoviral polynucleotide construct or a drug; and, optionally,
a monovalent or divalent salt or
a cross-linking agent or
a combination thereof; and
a water-based corona solution surrounding said core solution, said corona solution comprising:
at least one cation;
polycationic polymers; and
optionally, a targeting conjugate; or
a pharmaceutical composition thereof.

2. The composition of claim 1, wherein the monovalent or divalent salt is sodium chloride, calcium chloride or sodium sulfate.

3. The composition of claim 1, wherein the crosslinking agent is dextran polyaldehyde.

4. The composition of claim 1, wherein said targeting conjugate comprises a dextran-conjugated lectin or a dextran-conjugated glycan.

5. The composition of claim 1, wherein said polyanionic polymers are sodium alginate, pentasodium tripolyphosphate, kappa carrageenan, low-esterified pectin, polyglutamic acid, cellulose sulfate or chondroitin sulfate.

6. The composition of claim 1, wherein said polycationic polymers are polyvinylamine, spermine hydrochloride, protamine sulfate, polyethyleneimine, polyethyleneimine-ethoxylated, polyethyleneimine-epichlorhydrin modified, quarternized polyamide, polydiallyldimethyl ammonium chloride-co-acrylamide, chitosan glutamate, or pluronic F-68.

7. The composition of claim 1, wherein said cation is calcium chloride, potassium chloride or sodium chloride.

8. The composition of claim 1, wherein said polynucleotide is a gene.

9. The composition of claim 1, wherein said gene is a gene expressing an angiogenic growth factor.

10. The composition of claim 1, wherein said drug is an antiangiogenic growth factor.

11. The composition of claim 1, wherein said antiangiogenic growth factor is endostatin, thrombospondin 1 or thrombospondin 2 or a combination thereof.

12. The composition of claim 1, wherein, in said core solution, said polyanionic polymers are sodium alginate and cellulose sulfate, said salt is sodium chloride, said polynucleotide is a gene and said crosslinking agent is dextran polyaldehyde.

13. The composition of claim 1, wherein, in said core solution, said polyanionic polymers are pentasodium tripolyphosphate and kappa (iota)-carrageenan, said polynucleotide is a gene and said crosslinking agent is dextran polyaldehyde.

14. The composition of claim 1, wherein, in said core solution, said polyanionic polymers are sodium alginate and cellulose sulfate, said salt is sodium chloride or calcium chloride, said polynucleotide is a gene and said crosslinking agent is dextran polyaldehyde; and
wherein, in said corona solution, said polycations are spermine hydrochloride, PMCG hydrochloride and F-68, said cation is calcium chloride and said targeting conjugate is a dextran-conjugated lectin or a dextran-conjugated glycan.

15. The composition of claim 14, wherein, in said core solution, said polyanionic polymers are sodium alginate and cellulose sulfate, said salt is sodium chloride and said polynucleotide is a gene; and
wherein, in said corona solution, said polycations are spermine hydrochloride, PMCG hydrochloride and F-68, said cation is calcium chloride and said targeting conjugate is a dextran-conjugated lectin or a dextran-conjugated glycan.

16. The composition of claim 14, wherein, in said core solution, said polyanionic polymers are sodium alginate and cellulose sulfate, said salt is sodium chloride and said polynucleotide is a gene; and
wherein, in said corona solution, said polycations are spermine hydrochloride, PMCG hydrochloride and F-68 and said cation is calcium chloride.

17. The composition of claim 1, wherein, in said core solution, said polyanionic polymers are pentasodium tripolyphosphate and kappa (iota)-carrageenan, said salt is sodium chloride, said polynucleotide is a gene and said crosslinking agent is dextran polyaldehyde; and
wherein, in said corona solution, said polycations are chitosan glutamate and F-68, said cations are sodium chloride and/or calcium chloride and said targeting conjugate is a dextran-conjugated lectin or a dextran-conjugated glycan.

18. The composition of claim 17, wherein, in said core solution, said polyanionic polymers are pentasodium tripolyphosphate and kappa (iota)-carrageenan, said salt is sodium chloride and said polynucleotide is a gene; and
wherein, in said corona solution, said polycations are chitosan glutamate and F-68, said cation is calcium chloride and said targeting conjugate is a dextran-conjugated lectin or a dextran-conjugated glycan.

19. The composition of claim 17, wherein, in said core solution, said polyanionic polymers are pentasodium tripolyphosphate and kappa (iota)-carrageenan, said polynucleotide is a gene and said crosslinking agent is dextran polyaldehyde; and
wherein, in said corona solution, said polycations are chitosan glutamate and F-68 and said cations are sodium chloride and calcium chloride.

20. The composition of claim 1, wherein individually said polyanionic polymers are present in a concentration of about 0.01 wt % to about 0.5 wt %.

21. The composition of claim 1, wherein monovalent or divalent salt is present in a concentration up to about 3 wt %.

22. The composition of claim 1, wherein the cross-linking agent is present in a concentration of about 0.01 wt % to about 0.1 wt %.

23. The composition of claim 1, wherein said adenoviral polynucleotide conjugate is present in a concentration of about 0.01 wt % to about 0.1 wt %.

24. The composition of claim 1, wherein individually said polycationic polymers are present in a concentration of about 0.01 wt % to about 1.0 wt %.

25. The composition of claim 1, wherein said cation is present in a concentration of about 0.1 wt % to about 3 wt %.

26. The composition of claim 1, wherein said targeting conjugate is present in a concentration of about 0.01 wt % to about 0.1 wt %.

27. The composition of claim 1, said composition forming a nanoparticulate structure, a microcapsular structure or a polymeric sheet structure.

28. A method of delivering a polynucleotide or a drug to a human or a non-human animal to treat a pathophysiological state therein comprising the step of:
administering the composition of claim 1 to the human or the non-human animal, said composition containing a pharmacologically effective amount of said polynucleotide or of said drug.

29. A composition comprising:
a water-based core solution comprising:
sodium alginate;
cellulose sulfate;
an adenoviral gene construct;
sodium chloride or calcium chloride; and
optionally, dextran polyaldehyde; and
a water-based corona solution surrounding said core, said corona solution comprising:
spermine hydrochloride;
PMCG hydrochloride;
pluronic F-68;
calcium chloride; and
optionally, a dextran-conjugated lectin or a dextran-conjugated glycan; or
a pharmaceutical composition thereof.

30. The composition of claim 29, wherein, in said core solution, concentrations of sodium alginate and cellulose sulfate are individually about 0.05 wt-%, concentration of sodium chloride is about 2.0 wt-% or concentration of calcium chloride is about 1.0 wt-%, concentration of said adenoviral gene construct is about 0.01 wt-% to about 0.1 wt-%, and concentration of said optional dextran polyaldehyde is about 0.01 wt-% to about 0.1 wt-%.

31. The composition of claim 29, wherein, in said corona solution, concentrations of spermine hydrochloride and PMCG hydrochloride are individually about 0.05 wt-%, concentration of pluronic F-68 is about 1 wt-%, concentration of calcium chloride is about 0.05 wt-%, and concentration of said optional dextran-conjugated lectin or dextran-conjugated glycan is about 0.01 wt-% to about 0.1 wt-%.

32. The composition of claim 29, wherein said composition forms a nanoparticulate structure, a microcapsular structure or a polymeric sheet structure.

33. A method of delivering a polynucleotide or a drug to a human or a non-human animal to treat a pathophysiological state therein comprising:
administering the composition of claim 29 to the human or the non-human animal, said composition containing a pharmacologically effective amount of said polynucleotide or of said drug.

34. A composition comprising:
a water-based core solution comprising:
pentasodium tripolyphosphate;
kappa (iota)-carrageenan;
an adenoviral gene construct; and, optionally,
sodium chloride or
dextran polyaldehyde or
a combination thereof; and
a water-based corona solution surrounding said core, said corona solution comprising:
chitosan glutamate;
pluronic F-68;
calcium chloride;
optionally, sodium chloride; and
optionally, a dextran-conjugated lectin or a dextran-conjugated glycan; or
a pharmaceutical composition thereof.

35. The composition of claim 34, wherein, in said core solution, concentrations of pentasodium tripolyphosphate and kappa (iota)-carrageenan are individually about 0.01 wt-%, concentration of optional sodium chloride is about 2.0 wt-%, concentration of said adenoviral gene construct is about 0.01 wt-% to about 0.1 wt-%, and concentration of said optional dextran polyaldehyde is about 0.01 wt-% to about 0.1 wt-%.

36. The composition of claim 34, wherein, in said corona solution, concentrations of chitosan glutamate is about 0.05 wt-%, concentration of pluronic F-68 is about 1 wt-%, concentration of sodium chloride is about 1.0 wt-% and/or concentration of calcium chloride is about 0.01 wt-%, and concentration of said optional dextran-conjugated lectin or dextran-conjugated glycan is about 0.01 wt-% to about 0.1 wt-%.

37. The composition of claim 34, wherein said composition forms a nanoparticulate structure, a microcapsular structure or a polymeric sheet structure.

38. A method of delivering a polynucleotide or a drug to a human or a non-human animal to treat a pathophysiological state therein comprising the step of:
administering the composition of claim 34 to the human or the non-human animal, said composition containing a pharmacologically effective amount of said polynucleotide or of said drug.
